(19) United States
(12) Patent Application Publication
(10) Pub. No.: US 2013/0172624 A1
(43) Pub. Date: Jul. 4, 2013

(54) PROCESS FOR THE OXIDATION OF ALKYLAROMATIC HYDROCARBONS CATALYZED BY N-HYDROXY DERIVATIVES

(71) Applicants: Francesco Recupero, Milano (IT); Carlo Punta, Milano (IT); Lucio Melone, Montefusco (AV) (IT); Simona Prosperini, Santhia (VC) (IT); Nadia Pastori, Rho (MI) (IT)

(72) Inventors: Francesco Recupero, Milano (IT); Carlo Punta, Milano (IT); Lucio Melone, Montefusco (AV) (IT); Simona Prosperini, Santhia (VC) (IT); Nadia Pastori, Rho (MI) (IT)

(73) Assignee: Versalis S.p.A., San Donato Milanese (MI) (IT)

(21) Appl. No.: 13/805,478

(22) PCT Filed: Jun. 20, 2011
(86) PCT No.: PCT/IB2011/001411

§ 371 Date: Mar. 7, 2013

(30) Foreign Application Priority Data
Jun. 25, 2010 (IT) MI2010A001159

(51) Int. Cl. C07C 45/27

(52) U.S. Cl. CPC C07C 45/27
USPC 568/303

(57) ABSTRACT

Process for the oxidation of alkylaromatic hydrocarbons to hydroperoxide catalyzed by N-hydroxy derivatives in the presence of a solvent which includes recovering the catalyst from the oxidation mixture by the possible removal of the solvent by distillation and/or cooling of the oxidation mixture, with the consequent precipitation and filtration of the N-hydroxy-derivative catalyst, and adsorption with non-basic adsorbing solids for the substantially complete recovery of the catalyst.

### FIELD OF THE INVENTION

The present invention relates to a process for the oxidation of alkylaromatic hydrocarbons catalyzed by N-hydroxy derivatives.

More specifically, the alkylaromatic hydrocarbons are converted to the corresponding hydroperoxide by selective aerobic oxidation under mild conditions in the presence of a catalytic system based on N-hydroxy derivatives.

The catalyst is recovered from the reaction mixture at the end of the oxidation process by precipitation and/or adsorption on adsorbing resins, subsequently washed with suitable polar solvents, by means of a process described hereunder.

The hydroperoxides of alkylaromatic hydrocarbons are possibly transformed into phenol and carbonyl compounds, for example C₂-C₅₀, preferably C₂-C₂₀, (cyclo)aliphatic and aromatic aldehydes and ketones, hereinafter carbonyl compounds, in a process catalyzed by acids.

### PRIOR ART TO THE INVENTION

The industrial production of phenol is based on the Hock process in which the autoxidation of cumene is effected to the respective hydroperoxide together with the decomposition of the latter by acid catalysis into phenol and acetone (Ullman's Encyclopedia of Industrial Organic Chemicals, Vol. A9, 1958, 225, Wiley-VCH). The most complex phase which most widely influences the whole process is the autoxidation, in which the hydroperoxide formed acts in turn as radical chain initiator at high temperatures, generating the cumyloxy radical by thermal decomposition. The selectivity in the formation of the hydroperoxide decreases to the extent in which a relatively high conversion is reached, due to a greater decomposition of the hydroperoxide itself. Furthermore, the methyl radical, formed in the β-scission of the cumyloxy radical, is oxidized under the reaction conditions to formic acid. The latter catalyzes the decomposition of the hydroperoxide to phenol, which inhibits the oxidation process. In industrial processes, it is therefore necessary to operate in the presence of a base in order to neutralize the carboxylic acid. This problem also arises in the oxidation of other alkyl aromatic derivatives, for example in the oxidation of sec-butylbenzene in which significant quantities of acetic acid are formed (WO 2009/058527).

In order to eliminate or reduce these disadvantages, several expedients have been taken into consideration, such as the use of suitable metallic complexes as catalysts or co-catalysts, which increase the conversion rate and allow lower temperatures to be used, at which the hydroperoxide is more stable (Ishii, Y. at al. J. Mol. Catalysis. A, 1987, 117, 123). The higher thermal stability of the hydroperoxides at low temperatures, however, is negatively balanced by the redox decomposition caused by the metallic salts. These catalytic systems have consequently proved to be inadequate for the preparation of hydroperoxides.

New catalytic systems have recently been proposed for the aerobic oxidation of cumene and other alkylaromatic hydrocarbons, based on the use of N-hydroxyimides and N-hydroxysulfamides associated with radical initiators, such as peroxides and azo-derivatives which operate without metal salts (Ishii, Y. at al. Adv. Synth. Catal. 2001, 343, 809 and 2004, 346, 199; Sheldon, R. A. at al. Adv. Synth. Catal. 2004, 346, 1051; Levin, D. at al. WO 2007/073916 A1; U.S. Pat. Nos. 6,852,893; 6,720,462). N-hydroxyphthalimide, which can be easily obtained from low-cost industrial products (phthalic anhydride and hydroxylamine) is of particular interest (Minisci, F. et al. J. Mol. Catal. A, 2003, 63, 204 and 2006, 251, 129; Recupero, F. and Punta C., Chem. Rev. 2007, 107, 3800-3842).

In the presence of N-hydroxyphthalimide (NHPI), aldehydes have proved to have a considerable activity for the production of hydroperoxide of alkylaromatic compounds under mild aerobic conditions, with a high conversion and selectivity (Minisci et al. WO 08/037,435; Minisci et al. WO 09/115,275).

The use of N-hydroxy-derivatives has definite advantages with respect to non-catalyzed autoxidations, but also some disadvantages deriving from the decomposition of the initiators.

The Applicants have recently found that N-hydroxyphthalimide can catalyze the peroxidation of alkylaromatic compounds under mild conditions if the aerobic oxidation is carried out in the presence of a moderate amount of polar solvents (ketones, nitriles, esters, dialkyl carbonates and tertiary alcohols) which are completely stable under the operative conditions (WO 2009/115276). The polar solvent has a key function in favouring the dissolution of the organic catalyst, otherwise essentially insoluble under the operative conditions necessary for guaranteeing a high selectivity to hydroperoxide (temperature preferably lower than 110° C.). Under the same operating conditions, in the absence of N-hydroxy-derivative, there is no significant reaction.

In the peroxidation of cumene, acetone is obtained as co-product during the acid decomposition of the hydroperoxide to phenol.

The demand for phenol, however, is constantly growing with respect to that for acetone. There is consequently a growing interest in processes for the production of phenol which avoid the formation of acetone.

The peroxidation of sec-butylbenzene is interesting as the cost of propylene, with respect to that of butenes, is continuously increasing and the offer is lower than the market request.

Cyclohexylbenzene is even more interesting. It has also been possible to effect the peroxidation process on this derivative with the method object of the present invention. The corresponding cyclohexanone, obtained together with phenol, is of great industrial interest for the production of caprolactone (precursor of nylon 6) and adipic acid. Furthermore, the possibility of converting phenol to cyclohexanone by hydrogenation, and cyclohexanone to phenol by dehydrogenation (Sheldon et al. Tetrahedron 2002, page 9055) allows the production to be programmed on the basis of the variation in the market requests for the two products.

The selectivity of this process to hydroperoxide is extremely high.

The N-hydroxy derivative, in particular the most convenient N-hydroxyphthalimide, remains unaltered, but must be recovered at the end of the oxidation process, before the hydroperoxide is subjected to scission. Most of it is extracted from the reaction mixture by crystallization and filtration, after removal of the polar solvent by distillation and subsequent cooling. A percentage of catalyst, however, which varies on the basis of the nature of the alkylaromatic hydrocarbons subjected to oxidation, remains in the solution, which is made more polar by the presence of the hydroperoxide formed. Various solutions have been proposed for recovering the catalyst quantitatively. Already in the previous patents (WO 2009/115275 and WO 2009/115276), the Applicants have demonstrated the possibility of recovering further quantities of catalyst by means of aqueous extraction. The volumes of water necessary for a quantitative recovery of the catalyst, however, are high and consequently the procedure is difficult to be applied.

More recently Exxon Mobil has described the possibility of removing, at least partially, the catalyst from the reaction mixture by basic aqueous extraction (WO 2009/025939) or by treatment of the effluent with a solid adsorbent having basic properties (WO 2009/058527). In this way, the slightly acid characteristics of the catalyst can be exploited. This type of approach is particularly effective in the extraction phase. In the former case, however, a subsequent acid treatment of the aqueous solution is required to complete the recovery of the catalyst with a consequent production of high quantities of inorganic salts. Analogously, an effective removal of the catalyst from the basic adsorbing solids requires either considerable volumes of polar solvents or the use of acid solvents, with the consequent necessity of regenerating the resins and recovering the catalyst in the non-salified form.

The present invention describes the use of materials consisting of non-basic adsorbing resins for the extraction of the catalyst from the reaction mixture, upstream of the scission process, by physical adsorption exclusively. This process can be possibly performed after removal, also partial, of the solvent and precipitation, partial, of the catalyst which can thus be collected by filtration.

After adsorption, the catalyst can then be recovered and recycled by simple washing of the adsorbing resins with minimum amounts of polar solvents.

### DESCRIPTION OF THE INVENTION

The object of the present invention, better described in the enclosed claims, therefore relates to a process for the oxidation of alkylaromatic hydrocarbons which includes:

1) the selective aerobic oxidation of alkylaromatic hydrocarbons to the corresponding hydroperoxide, catalyzed by N-hydroxy derivatives in the presence of polar solvents and possibly water (co-solvent);
2) removal of the N-hydroxy-derivative catalyst from the reaction mixture, by means of:

precipitation and filtration, following the removal of the polar solvent, and subsequent physical adsorption on suitable adsorbing resins, described hereunder; or direct physical adsorption on suitable adsorbing resins;

3) recovery of the catalyst by washing the filters and/or adsorbing resins with suitable polar solvents, possibly containing variable percentages of water;
4) possible scission of the hydroperoxide of the alkylaromatic hydrocarbon to phenol and carbonyl compound by homogeneous and heterogeneous acid catalysts.

### Oxidation of the Alkylaromatic Hydrocarbon:

The alkylaromatic hydrocarbon is converted to the corresponding hydroperoxide by means of aerobic oxidation in the presence a catalytic system, which includes N-hydroxyimides or N-hydroxysulfamides, preferably N-hydroxyphthalimide, associated with a polar solvent, in the presence or absence of initiators based on aliphatic or aromatic aldehydes. The temperature does not exceed 130° C., and ranges for example from 50 to 110° C., preferably from 80 to 100° C. if operating without an initiator, from 50 to 80° C. if operating in the presence of an aldehyde initiator. The polar solvent can be a C₃-C₂₀ acyclic, cyclic or aromatic ketone (for example acetone, methylethylketone, 2-pentanone, 3-pentanone, methyl-t-butylketone, cyclopentanone), preferably the same coming from the acid decomposition of the hydroperoxide, or other solvents such as nitriles, esters, tertiary alcohols, dialkyl carbonates, which are also stable under the reaction conditions. A quantity of water ranging from 0.1 to 10% by weight with respect to the polar solvent, for example ranging from 0.1 to 5%, can possibly be added to polar solvent, to favour the solubility of the catalyst in the reaction medium.

The quantity of N-hydroxy-derivative catalyst ranges from 0.1 to 10% in moles, for example from 0.5 to 5%, preferably from 1 to 2% in moles, with respect to the starting alkylaromatic hydrocarbon.

The ratio between the volume of polar solvent with respect to the volume of the alkylaromatic hydrocarbon is preferably within the range of 5:1 to 1:20.

The reaction is carried out with oxygen or air or N₂/O₂ mixtures having a ratio between N₂ and O₂ ranging from 10:1 to 1:10, operating at pressures ranging from 1 to 20 bar.

Under the same operative conditions, in the absence of N-hydroxyhpthalimide, the reaction does not take place to a significant degree.

When the process is carried out in the presence of an aldehyde initiator, the quantities of aldehydes used as precursors of the activators generated in situ, preferably range from 0.2% to 10%, with respect to the starting alkylaromatic hydrocarbon.

The alkylaromatic hydrocarbons include C₈-C₅₀, preferably C₈-C₂₀ hydrocarbons, for example ethylbenzene, cumene, cyclohexylbenzene, diphenylmethane and sec-butylbenzene.

### Treatment of the Oxidation Mixture:

The N-hydroxy derivative, in particular the most convenient N-hydroxyphthalimide, remains unaltered, but must be recovered at the end of the oxidation process, before the hydroperoxide is subjected to scission.

The oxidation mixture can be treated directly with adsorbing resins or it can be previously subjected to distillation to remove the polar solvent, and possibly water, and then cooled to a temperature ranging from −20 to 100° C., preferably from 0 to 60° C., for example 25° C. In this case, most of the catalyst is recovered from the reaction mixture by precipitation and filtration, in a percentage ranging from 50 to 90%, for example 80%, with respect to the quantity of N-hydroxyphthalimide initially introduced into the reactor. The quantity of catalyst which precipitates depends on the nature of the alkylaromatic hydrocarbon, the cooling temperature of the reaction mixture and conversion percentage to the corresponding hydroperoxide, which influences the nature of the oxidation mixture, increasing its polarity.

The oxidation mixture as such or concentrated and filtered, still containing a variable amount of N-hydroxy-derivative completely dissolved in solution, is treated with a non-basic adsorbing resin which is effective in removing the catalyst totally and can be easily regenerated, for example by washing with a polar solvent. In this way, an oxidation effluent is obtained, which is ready for the scission process and the N-hydroxy-derivative catalyst is quantitatively recovered and can be routed for recycling.

The selected adsorbing solids consist of non-basic resins, including ion exchange resins, which are capable of adsorbing the N-hydroxy-derivative catalyst by physical interaction, possibly also by inclusion. Resins which can be used in the present invention are therefore resins with a phenol, acrylic, styrene, styrene-divinylbenzene structure, for example, anionic resins, containing for example quaternary ammonium salts, cationic resins and also other neutral resins which give simple physical adsorption of the N-hydroxy-derivative catalyst.

Resins having basic properties and therefore capable of adsorbing the catalyst, which is weakly acid, by acid-base interaction, are not included. Adsorbing solids having basic properties are in fact particularly effective in the adsorption phase of the catalyst, but they do not easily release the same by simple washing with polar solvents, and therefore require acid treatment and subsequent regeneration.

The selection of suitable resins which adsorb the catalyst by simple physical interaction surprisingly guarantees high efficiency both in the adsorption and in the desorption phases and for the final recovery of the N-hydroxy-derivative. Non-basic ion exchange resins are also optimum for this purpose.

Basic anion exchange resins (OH⁻, HCO₃⁻ and CO₃²⁻ counter-ions) can be selected and used in the process, object of the present invention, due to their structural characteristics, but after treatment with saline or acid solutions which cancel the base characteristics, thanks to the substitution of the counter-ion bound to the resin. The selection of the counter-ion on the resin allows its structure to be modulated, improving the performance in the adsorption and/or desorption phase.

The resins of choice may require washing treatment and regeneration before being used for the purpose illustrated herein.

The operative conditions in the adsorption phase include a temperature ranging from 20 to 90° C. The adsorption temperature is selected on the basis of the amount of N-hydroxy derivative to be recovered: for recovering the 100% on an adsorbing resin, high temperatures are adopted. For recovering on an adsorbing resin downstream of a cooling and filtration process, lower temperatures are used. The temperature in the adsorption phase is selected on the basis of the quantity of N-hydroxy derivative which is to be precipitated and recovered by filtration. The contact time in the adsorption phase ranges from 1 minute to 2 hours, for example 1 hour. The adsorption can also takes place by passing the oxidation mixture in a column filled with the adsorbing solid.

The N-hydroxy-derivative catalyst is then removed from the adsorbing solid by washing with a polar solvent, preferably the solvent used in the oxidation process. The polar solvent can be a C₃-C₂₀ acyclic, cyclic or aromatic ketone (for example acetone, methylethylketone, 2-pentanone, 3-pentanone, methyl-t-butylketone, cyclopentanone), preferably the same deriving from the acid decomposition of the hydroperoxide, or other solvents such as nitriles, esters, tertiary alcohols, dialkyl carbonates, which are also stable under the reaction conditions or a mixture of two or more of these. The desorption phase takes place at a temperature ranging from 20 to 130° C., preferably from 30 to 100° C., for example 60° C. The quantity of polar solvent used varies in relation to the nature of the solvent, the quantity of catalyst to be recovered and the quantity and quality of adsorbing resin subjected to washing.

### Scission of the Hydroperoxide to Phenol and Ketone

The hydroperoxide of the alkylaromatic hydrocarbon, obtained in the oxidation phase according to the procedure described above, is possibly transformed to phenol and carbonyl compound by contact with an acid catalyst in homogeneous or heterogeneous phase.

After removing the polar solvent, used in the oxidation reaction, and after recovering the catalyst, the reaction mixture is introduced into the scission reactor, preferably at a concentration of hydroperoxide obtained in the oxidation process, ranging for example from 20 to 30%. Alternatively, the oxidation reaction mixture can be concentrated to up to 85% by weight of hydroperoxide before being introduced into the scission reactor, by removal of the alkylaromatic hydrocarbon at reduced pressure. Alternatively, the oxidation reaction mixture can be diluted with inert solvent which favours the removal of the heat developed.

The scission reaction can be carried out in a distillation unit. The process is carried out at a temperature ranging from 0 to 150° C., preferably from 20 to 80° C. The pressure preferably ranges from 1 to 20 bar.

Protic acids can be used as homogeneous catalysts (for example sulfuric acid, phosphoric acid, hydrochloric acid, p-toluenesulfonic acid) or Lewis acids (for example ferric chloride, zinc chloride, boron trifluoride). Acid zeolites such as, for example, beta zeolites, zeolites Y, X, ZSM-5, ZSM-12 or mordenite, can be used as heterogeneous catalysts.

The mixture deriving from the scission is subjected to distillation to recover the carbonyl compound, the phenol and the non-reacted alkylaromatic hydrocarbon.

The following examples are provided for illustrative but non-limiting purposes for the process of the present invention.

### Example 1

### Activation of the Adsorbing Resins

The resins Amberlyst A26 and A26OH, basic resins having an OH⁻ counterion, were washed first with water then with acetone to remove the free amines of which they were impregnated.

Part of these resins was treated with an aqueous solution of hydrochloric acid 2M (300 mL of solution for three times) obtaining the resins indicated with A26(Cl) and A26OH(Cl); another part was treated with an aqueous solution of p-toluenesulfonic acid 0.5 M (300 mL solution for three times) obtaining the resins indicated with A26(Ts) and A26OH(Ts). Each resin was finally washed abundantly with water until a neutral pH was obtained and with acetone. They were then dried in air and under forced vacuum.

The resin Amberlite IRA-400(Cl) having Cl⁻ as counterion was used as such without any treatment.

The acid resin Amberlyst A15, having H⁺ as counterion, was treated with an aqueous solution of NaOH 2M (300 mL for 3 times). The resin was washed abundantly with water until a neutral pH was obtained and then with acetone, and was left to dry in air and under forced vacuum obtaining the resin A15(Na) having Na⁺ ions as counterions.

The adsorbing resins Amberlyst XAD761 and XAD7HP were washed abundantly with water, initially slightly acidulated with HCl in order to remove the alkaline carbonates with which they were impregnated for protective purposes, finally washed with acetone and left to dry first in air and then under forced vacuum.

### Example 2

### Oxidation of Cumene

A solution composed of 400 mL of cumene (2.870 mmoles), 150 mL of acetonitrile and 28.7 mmoles (4.7 g) of N-hydroxyphthalimide is stirred at 60° C. for 6 hours in an oxygen atmosphere at a pressure of 1 bar. ¹H-NMR analysis of the reaction mixture showed a conversion of cumene equal to 35% with a selectivity to cumyl hydroperoxide of 99% (result confirmed through iodometric titration, GC-MS analysis in the presence of an internal standard after reduction of the hydroperoxide to the corresponding alcohol with PPh₃ and HPLC analysis of the reaction mixture without any treatment.

### Example 3

### Removal of the N-Hydroxyphthalimide Catalyst from the Reaction Mixture by Precipitation

The solvent acetonitrile was removed by distillation from the reaction mixture according to Example 2 until a final volume of about 400 mL was reached. The residual mixture was cooled to room temperature in order to favour the precipitation of the N-hydroxy-derivative catalyst.

After 1 hour, about 3.750 g of NHPI were recovered, equal to 80%⁻ of the catalyst initially introduced into the reactor.

### Example 4

### Removal of the N-Hydroxyphthalimide Catalyst Using Adsorbing Resins

The reaction mixture having a volume of about 400 mL, already subjected to recovery of the N-hydroxyphthalimide by precipitation, after removal of the solvent, as described in Example 3, and containing a residue of N-hydroxyphthalimide equal to about 950 mg (corresponding to a concentration of about 2.38 mg/mL) was divided into samples, each of 40 mL. Each sample was put in contact with different adsorbing solids, previously treated as described in Example 1, for 1 hour. All the experiments were performed at room temperature (25° C.). The percentage of N-hydroxyphthalimide adsorbed was determined by means of the HPLC technique, analyzing the mixture before and after the adsorption treatment.

The adsorbing resins used include: A26, A26OH, A26(Cl), A26OH(Cl), A26(Ts), A26OH(Ts), IRA-400(Cl), XAD 761, XAD 7HP and A15(Na). The results indicated below show that all the resins, including non-basic resins, substantially removed all the N-hydroxyphthalimide from the reaction mixture:
a. 5 g of Amberlyst A26, were put in contact with 40 mL of the solution obtained after the treatment according to Example 3, at room temperature for 1 hour, adsorbing a quantity of NHPI equal to about 94 mg (recovery higher than 98%).
b. 5 g of Amberlyst A26OH were put in contact with 40 mL of the solution obtained after the treatment according to Example 3, at room temperature for 1 hour, adsorbing a quantity of NHPI equal to about 94 mg (recovery higher than 98%).
c. 5 g of Amberlyst A26(Cl) were put in contact with 40 mL of the solution obtained after the treatment according to Example 3, at room temperature for 1 hour, adsorbing a quantity of NHPI equal to about 94 mg (recovery higher than 98%).
d. 5 g of Amberlyst A26OH(Cl) were put in contact with 40 mL of the solution obtained after the treatment according to Example 3, at room temperature for 1 hour, adsorbing a quantity of NHPI equal to about 94 mg (recovery higher than 98%).
e. 5 g of Amberlyst A26(Ts) were put in contact with 40 mL of the solution obtained after the treatment according to Example 3, at room temperature for 1 hour, adsorbing a quantity of NHPI equal to about 94 mg (recovery higher than 98%).
f. 5 g of Amberlyst A26OH(Ts) were put in contact with 40 mL of the solution obtained after the treatment according to Example 3, at room temperature for 1 hour, adsorbing a quantity of NHPI equal to about 94 mg (recovery higher than 98%).
g. 5 g of Amberlite IRA-400(Cl) were put in contact with 40 mL of the solution obtained after the treatment according to Example 3, at room temperature for 1 hour, adsorbing a quantity of NHPI equal to about 94 mg (recovery higher than 98%).
h. 8 g of XAD-761 were put in contact with 40 mL of the solution obtained after the treatment according to Example 3, at room temperature for 1 hour, adsorbing a quantity of NHPI equal to about 94 mg (recovery higher than 98%).
i. 10 g of XAD-7HP were put in contact with 40 mL of the solution obtained after the treatment according to Example 3, at room temperature for 1 hour, adsorbing a quantity of NHPI equal to about 94 mg (recovery higher than 98%).
j. 5 g of Amberlyst A15(Na) were put in contact with 40 mL of the solution obtained after the treatment according to Example 3, at room temperature for 1 hour, adsorbing a quantity of NHPI equal to about 94 mg (recovery higher than 98%).

### Example 5

### Recovery of the N-Hydroxyphthalimide Catalyst from the Adsorbing Resins by Washing with a Polar Solvent

For each adsorbing resin coming from the recovery treatment of N-hydroxyphthalimide described in Example 4, 5 g of sample were removed and subjected to the following treatment to verify the desorption efficiency: i) they were washed with n-hexane at room temperature (40 mL for 3 times) to remove the residue of reaction mixture; in this way the polar catalyst is not released; ii) they were subsequently subjected to regeneration by washing with 40 mL of acetonitrile (5 times for 1 hour) at room temperature. The percentage of N-hydroxyphthalimide desorbed was determined by means of the HPLC technique, analyzing the mixture after the desorption treatment.

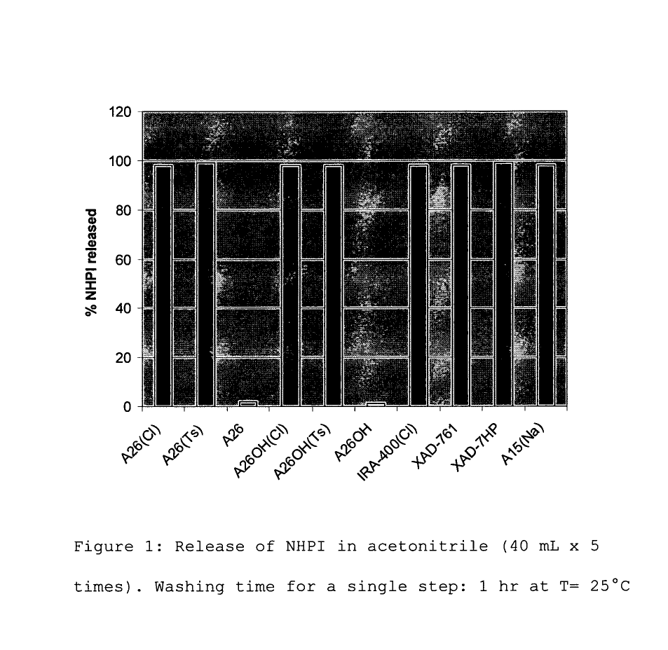

The results are indicated in FIG. 1 and show how, whereas the basic resins A26 and A26OH, on which the N-hydroxyphthalimide was adsorbed by acid-base interaction, only release traces of catalyst, the resins A26(Cl), A26OH(Cl), A26(Ts), A26OH(Ts), IRA-400(Cl), XAD 761, XAD 7HP and A15(Na), on which the adsorption of the N-hydroxyphthalimide takes place by simple physical interaction, quantitatively desorb the catalyst with a regeneration and recovery efficiency of the catalyst higher than 98%.

1. A process for oxidizing an alkylaromatic compound, the process comprising:
the selectively aerobically oxidizing of a C₈-C₅₀ alkylaromatic hydrocarbon to form a hydroperoxide, said oxidation catalyzed by at least one N-hydroxy derivative in the presence of at least one polar solvent and optionally water as a co-solvent;
recovering the at least one N-hydroxy derivative from an oxidation mixture, and optionally removing solvent by distillation, cooling, or both, from the oxidation mixture, with precipitation and filtration of the N-hydroxy-derivative; and
optionally splitting the hydroperoxide with at least one acid catalyst to form phenol and a carbonyl compound;
wherein the oxidation mixture, optionally concentrated, cooled and filtered, is treated with at least one non-basic adsorbing solid to complete recovery of the N-hydroxy derivative.

2. The process according to claim 1, wherein the N-hydroxy derivative is selected from the group consisting of N-hydroxy-phthalimide and N-hydroxy-saccharine.

3. The process according to claim 1, wherein the oxidation of the alkylaromatic hydrocarbon occurs at a temperature lower than 130° C.

4. The process according to claim 1, comprising selectively aerobically oxidizing the alkylaromatic hydrocarbon with oxygen, air or N₂/O₂ mixtures having a ratio between N₂ and O₂ ranging from 10:1 to 1:10, at a pressure ranging from 1 to 20 bar.

5. The process according to claim 1, wherein the oxidation of the alkylaromatic hydrocarbon occurs in the presence of at least one polar solvent selected from the group consisting of a ketone, a nitrile, an ester, a tertiary alcohol and a dialkyl carbonate ketones, nitriles, esters, tertiary alcohols, dialkyl carbonates.

6. The process according to claim 1, wherein from 0.1 to 10% by weight of water, with respect to the polar solvent, is added to the polar solvent.

7. The process according to claim 1, wherein the alkylaromatic hydrocarbon is selectivelly aerobically oxidized with from 0.1 to 10% in moles of the N-hydroxy derivative with respect to the alkylaromatic hydrocarbon.

8. The process according to claim 1, wherein a ratio of a volume of the polar solvent to a volume of the alkylaromatic hydrocarbon ranges from 5:1 to 1:20.

9. The process according to claim 1, wherein the oxidizing occurs in the presence of at least one initiator selected from the group consisting of a peracid, a dioxirane and an aldehyde.

10. The process according to claim 1, wherein the recovering of the at least one N-hydroxy derivative occurs by concentrating the oxidation mixture with partial or total removal of the polar solvent by distillation.

11. The process according to claim 10, wherein part of the N-hydroxy derivative is removed from the reaction mixture and cooled to a temperature lower than 100° C. by crystallization and filtration.

12. The process according to claim 11, wherein N-hydroxy derivative still present in the oxidation mixture is recovered by treatment with at least one non-basic adsorbing solid.

13. The process according to claim 1, wherein N-hydroxy derivative present in the oxidation mixture is recovered directly by treatment with at least one non-basic adsorbing solid, without any filtration process, at temperatures lower than 110° C.

14. The process according to claim 13, wherein the non-basic adsorbing solid is selected from the group consisting of a zeolite, an adsorbing resin, an ion exchange resin and mixtures thereof.

15. The process according to claim 1, wherein the N-hydroxy-derivative is recovered by washing a filter of the filtration with a polar washing solvent.

16. The process according to claim 12, wherein the N-hydroxy-derivative is recovered by washing the non-basic adsorbing solid with a polar washing solvent.

17. The process according to claim 15, wherein the polar washing solvent is at least one selected from the group consisting of a ketone, a nitrile, an ester, a tertiary alcohol, a dialkyl carbonate and mixtures thereof, with optional addition of a quantity of water ranging from 0.01 to 10% moles.

18. The process according to claim 15, wherein the polar washing solvent and the polar solvent of the oxidizing are the same solvent.

19. The process according to claim 1, wherein a polar solvent containing recovered N-hydroxy-derivative in solution is introduced into an oxidation reactor with the alkylaromatic hydrocarbon.

20. The process according to claim 1, comprising splitting the hydroperoxide of to form the phenol and the carbonyl compound in the presence of at least one acid catalyst selected from the group consisting of a protic acid and a Lewis acid.

21. The process according to claim 20, comprising splitting the hydroperoxide in the presence of at least one acid selected from the group consisting of sulfuric acid, phosphoric acid, hydrochloric acid, p-toluenesulfonic acid, Amberlyst, ferric chloride, zinc chloride, and boron trifluoride.

22. The process according to claim 20, comprising splitting the hydroperoxide in the presence of at least one heterogeneous acid selected from the group consisting of a beta zeolite, a Y zeolite, a X zeolite, ZSM-5, ZSM-12, and mordenite.

23. The process according to claim 1, comprising splitting the hydroperoxide to form the phenol and the carbonyl compound at a temperature ranging from 0 to 150° C.

24. The process according to claim 1, comprising splitting the hydroperoxide to form the phenol and the carbonyl compound at a pressure ranging from 1 to 20 bar.
